(19) United States
(12) Patent Application Publication
(10) Pub. No.: US 2018/0168836 A1
(43) Pub. Date: Jun. 21, 2018

(54) ANTICANNULATION WEB

(71) Applicant: W. L. Gore & Associates, Inc., Newark, DE (US)

(72) Inventors: Karl R. Chung, Phoenix, AZ (US); Martin J. Sector, Gilbert, AZ (US); Aaron Robison, Phoenix, AZ (US); Annette J. Dunn, Scottsdale, AZ (US)

(21) Appl. No.: 15/847,281

(22) Filed: Dec. 19, 2017

Related U.S. Application Data
(60) Provisional application No. 62/436,640, filed on Dec. 20, 2016.

(51) Int. Cl. A61F 2/954; A61M 25/01; A61M 25/00; A61M 25/09; A61F 2/07

(52) U.S. Cl. CPC A61F 2/954; A61M 25/0133; A61B 17/3421; A61M 25/09041; A61F 2/07; A61M 25/0082

(57) ABSTRACT

Various aspects of the present disclosure are directed toward a steerable endovascular graft delivery device including an anti-cannulation member. The delivery device generally includes a guide wire extension element, a steering wire, and a membrane. The steering wire is operable to deflect a distal end of the guide wire extension element, and the membrane is operable to prevent cannulation of the gap that is formed between the guide wire extension element and the steering wire when the distal end of the guide wire extension element is deflected.

### CROSS-REFERENCE TO RELATED APPLICATION

This application claims priority to Provisional Application No. 62/436,640, filed Dec. 20, 2016, which is herein incorporated by reference in its entirety.

### FIELD

The present disclosure relates generally to steerable medical devices, and more specifically to apparatuses, systems and methods for use with medical procedures involving the insertion of one or more devices involving steering functionality, such as endovascular devices.

### BACKGROUND

Endovascular procedures address a broad array of medical needs, including endovascular access, diagnosis, and/or repair through minimally invasive or relatively less invasive means than surgical approaches. Aortic aneurysms represent an example of one malady that has benefited from endovascular techniques. Each year thousands of lives are threatened by deadly aortic aneurysms. While conventional procedures for treating aortic aneurysms involve open surgery, some minimally invasive, catheter-based procedures have been developed in recent years. Some of these procedures involve placing an endovascular graft inside of the diseased aorta proximate the aneurysm such that blood flows through the endovascular graft, thereby avoiding the aneurysm. These procedures operate to isolate the aneurysm such that the aorta does not sustain further damage in the area of and surrounding the aneurysm.

Catheter-based procedures involve the endovascular delivery of one or more endovascular grafts. Typically, one or more guide wires are inserted into and routed through the patient's vasculature to a target site where an aneurysm is located. Delivery catheters with endovascular grafts are routed along the guide wires to the target site. Once properly positioned at the target site, the endovascular grafts are deployed.

In some cases, directing the guide wire to the target site within the vasculature is difficult due to the vasculature's tortuous nature. Some patient's vasculature is more tortuous than other's. Some recently developed catheter devices and systems provide physicians with the ability to manipulate the distal end of the catheter to assist in navigating the catheter through patient's vasculature. In some of these systems, a steering wire or tether is coupled to a distal end of a guide wire extension element such that when tension is applied to the steering wire (or tether), the guide wire extension element is forced to bend or otherwise deflect. This deflection results in a bow-and-string configuration of the steering wire and guide wire extension element (e.g., the guide wire extension element as the bow and the steering wire as the string). In such a bow-and-string configuration, an opening or void is created between the steering wire and the guide wire extension element where an intermediate portion of the steering wire separates from the guide wire extension element.

This void or opening between the steering wire and the guide wire extension element can lead to complications where the opening or void is subsequently cannulated or penetrated by one or more other guide wires, catheters, or endovascular grafts. Some examples involve the introduction of a guide wire through a contralateral leg of a bifurcated endovascular graft for placement of an additional endovascular graft. Physicians utilizing fluoroscopy generally have only a two dimensional display of the target area within which they are working and therefore cannot definitively tell whether the void between the steering wire and the deflected guide wire extension element has been cannulated or penetrated by another guide wire or instrument. Where cannulation of the void has occurred, patients are exposed to risks, such as dislodgement of the endovascular graft as the catheter is subsequently withdrawn from the endovascular graft.

### SUMMARY

According to one aspect of the disclosure, an steerable apparatus for insertion into a body includes a guide wire extension element, a steering wire, and a membrane secured to the steering wire and the guide wire extension element. In some examples, the steering wire operates to cause a portion of the guide wire extension element to deflect away from the steering wire. In some examples, a void is formed between the guide wire extension element and the steering wire when the guide wire extension element is deflected away from the steering wire, and the membrane operates to cover the void to facilitate anticannulation of the void.

In some examples, the membrane is elastic and is configured to stretch to accommodate a change in curvature of the guide wire extension element as it deflects away from the steering wire. In some examples, the membrane is pre-formed based on a profile the guide wire extension element and the steering wire adopt when the steering wire operates to cause the guide wire extension element to deflect away from the steering wire.

In some examples, a distal end of the steering wire is coupled to the guide wire extension element. In some examples, applying a tension to the steering wire causes a portion of the guide wire extension element to deflect away from the steering wire.

In some examples, the steerable apparatus further includes a tubular element, wherein the steering wire and the guide wire extension element extend through a lumen of the tubular element and project distally from a distal end of the tubular element. In some examples, the guide wire extension element has a lumen extending through its interior that is configured to accommodate a guide wire such that the guide wire extension element can be guided along the guide wire. In some examples, the steerable apparatus further includes an olive coupled to a distal end of the guide wire extension element. Specifically, in some examples, a distal end of the steering wire is coupled to a portion of the olive.

In some examples, the membrane is coupled to one of the steering wire and the guide wire extension element. In some examples, the membrane is folded over the guide wire extension element and the steering wire and attached to itself. For instance, in some examples, the membrane is wound around the guide wire extension element and the steering wire and attached to itself.

In some examples, the membrane is formed of a high strength film.

According to one aspect of the disclosure, a method of manufacturing a steerable apparatus for insertion into a body includes providing a steerable catheter delivery device including a guide wire extension element and a steering wire coupled to the guide wire extension element such that a force applied to the steering wire causes the guide wire extension element to deflect away from the steering wire to form a void between the guide wire extension element and the steering wire. The method further includes coupling a membrane to the steerable catheter delivery device such that the membrane spans the void upon deflecting the guide wire extension element away from the steering wire such that the membrane operates to facilitate anticannulation of the void.

According to one aspect of the disclosure, an endovascular delivery method includes delivering a steerable guide wire assembly to a target site within a patient. In some examples, the steerable guide wire assembly of this endovascular delivery method includes a guide wire extension element, a steering wire, and a membrane in communication with the steering wire and the guide wire extension element. The endovascular delivery method further includes radially displacing a portion of the guide wire extension element from the steering wire such that the guide wire extension element defines a curved portion forming a void between the curved portion and the steering wire. The membrane operates to span the void to facilitate anticannulation of the void.

In some examples, the guide wire extension element is radially displace from the steering wire by applying a tension to the steering wire such that the guide wire extension element defines the curved portion. In some examples, the endovascular delivery method further includes releasing the tension to the steering wire to eliminate the separate of the guide wire extension element and the steering wire.

### BRIEF DESCRIPTION OF THE DRAWINGS

The accompanying drawings are included to provide a further understanding of the disclosure and are incorporated in and constitute a part of this specification, illustrate examples, and together with the description serve to explain the principles of the disclosure.

FIG. 1 is an illustration of a steerable endovascular graft delivery device in an unsteered state consistent with various aspects of the present disclosure.

FIG. 2 is an illustration of a steerable endovascular graft delivery device in a steered state consistent with various aspects of the present disclosure.

FIG. 3 is an illustration of a steerable endovascular graft delivery device in a steered state consistent with various aspects of the present disclosure.

FIG. 4A is an illustration of a steerable endovascular graft delivery device consistent with various aspects of the present disclosure.

FIG. 4B is an illustration of a steerable endovascular graft delivery device consistent with various aspects of the present disclosure.

FIGS. 5A-5C illustrate the membrane performance of a steerable endovascular graft delivery device consistent with various aspects of the present disclosure.

FIGS. 6A-6C illustrate the membrane performance of a steerable endovascular graft delivery device consistent with various aspects of the present disclosure.

FIGS. 7A-7C illustrate the membrane performance of a steerable endovascular graft delivery device consistent with various aspects of the present disclosure.

FIG. 8 is a cross-sectional illustration of a membrane attachment with a steerable endovascular graft delivery device consistent with various aspects of the present disclosure.

FIG. 9 is a cross-sectional illustration of a membrane attachment with a steerable endovascular graft delivery device consistent with various aspects of the present disclosure.

FIG. 10 is a cross-sectional illustration of a membrane attachment with a steerable endovascular graft delivery device consistent with various aspects of the present disclosure.

FIG. 11 is a cross-sectional illustration of a membrane attachment with a steerable endovascular graft delivery device consistent with various aspects of the present disclosure.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting. In describing various embodiments, the term distal is used to denote a position along an exemplary device proximate to or alternatively nearest to the treatment region within a patient's body. The term proximal is used to denote a position along the exemplary device proximate to or alternatively nearest to the user or operator of the device.

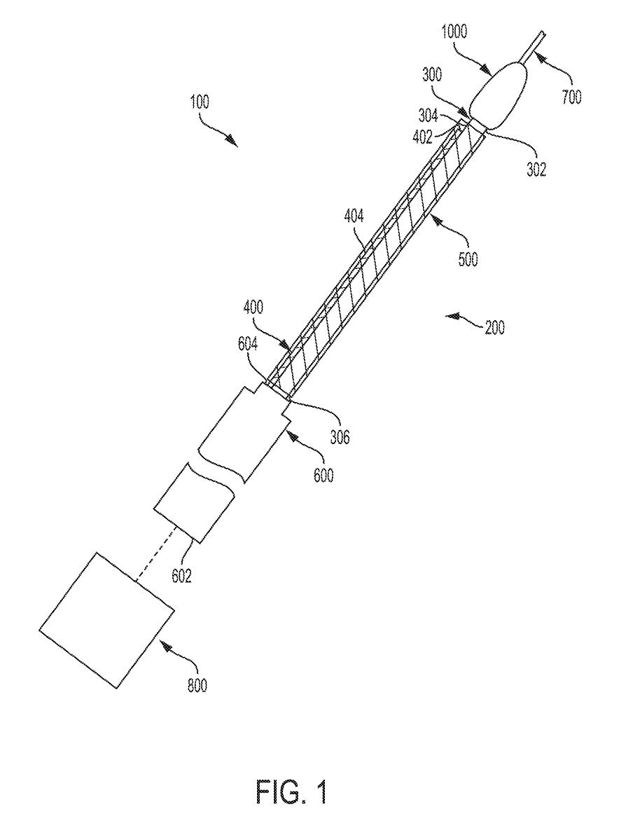

Various aspects of the present disclosure are directed toward a steerable medical device including a membrane or other feature that operates to prevent cannulation of the void created when a portion of a steering wire separates from a guide wire extension element. Although various examples are provided in the context of stent graft delivery applications, those examples should be understood to also relate to a wide variety of applications in addition to stent graft delivery. An exemplary steerable endovascular graft delivery system 100 is illustrated in FIG. 1. The delivery system 100 includes a catheter assembly 200 including of a guide wire extension element 300, a steering wire 400, and a membrane 500. As shown, the delivery system 100 optionally includes a tubular element 600, a guide wire 700 and a control mechanism 800. As shown, the control mechanism 800 is coupled to a proximal end 602 of the tubular element 600 and operates to provide control of the catheter assembly 200. The control mechanism 800 is optionally integral with one or more of the above-mentioned delivery system components.

As shown, the guide wire extension element 300 is a longitudinally extending structure and is configured for insertion within the body of a patient. The guide wire extension element 300 can be any longitudinally extending structure with or without a lumen extending therethrough. Thus, the guide wire extension element 300 may include but is not limited to tubes with lumens, solid rods, hollow or solid wires, hollow or solid stylets, metal tubes (e.g., hypotubes), polymer tubes, pull cords or tethers, fibers, filaments, electrical conductors, radiopaque elements, radioactive elements and radiographic elements. The guide wire extension element 300 can be of any material and can have any cross-sectional shape including but not limited to profiles that are circular, oval, triangular, square, polygon-shaped or randomly-shaped.

In some examples, the guide wire extension element 300 is a long hollow tube having a lumen extending from a proximal end (not illustrated) to a distal end 302 and is configured to accommodate the guide wire 700. In some examples, the proximal end of the guide wire extension element 300 is concealed within the tubular element 600. In some embodiments, the proximal end of the guide wire extension element 300 extends from the tubular element 600 such that it can be manually manipulated by an operator. In some embodiments, the proximal end of the guide wire extension element 300 extends from the tubular element 600 to the control mechanism 800 such that it can be manipulated by the control mechanism 800. In some embodiments, the proximal end of the guide wire extension element 300 extends from the control mechanism 800 such that it can be manually manipulated by an operator. The guide wire 700 extends through guide wire extension element 300 and projects distally from the distal end 302 of guide wire extension element 300. In some examples, the guide wire extension element 300 directs or otherwise guides the catheter assembly 200 to a target site within a patient's vasculature.

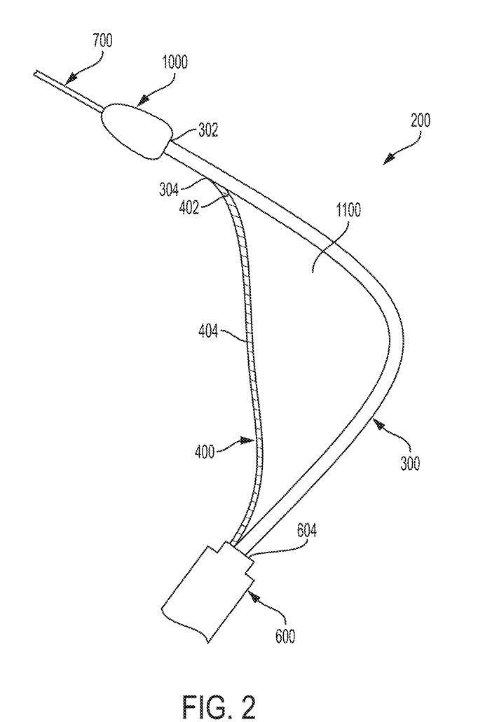

In various examples, the guide wire extension element 300 is flexible in that it can be manipulated to bend along its length (see e.g., FIG. 2). As shown, one or more steering wires, such as steering wire 400 are positioned proximate guide wire extension element 300 and facilitate the bending of the guide wire extension element 300. The steering wire 400 optionally extends along a length of the guide wire extension element 300. As shown, the steering wire includes a distal end portion 402, a proximal end portion (not illustrated), and an intermediate portion 404 situated between the proximal end portion and the distal end portion 402. In some examples, like the guide wire extension element, a proximal end of the steering wire 400 is concealed within the tubular element 600. In some embodiments, the proximal end of the steering wire 400 extends from the tubular element 600 such that it can be manually manipulated by an operator. In some embodiments, the proximal end of the steering wire 400 extends from the tubular element 600 to the control mechanism 800 such that it can be manipulated by the control mechanism 800. In some embodiments, the proximal end of the steering wire 400 extends from the control mechanism 800 such that it can be manually manipulated by an operator.

In various examples, the steering wire 400 is anchored, fixed, coupled, adhered, or otherwise fastened to the guide wire extension element 300. In some examples, the distal end portion 402 of the steering wire 400 is anchored, fixed, coupled, or otherwise fastened to the guide wire extension element 300 at a distal position along the guide wire extension element 300. In some examples, the steering wire 400 is coupled to the guide wire extension element 300 at its distal end 302. In some examples, the steering wire 400 is coupled to the guide wire extension element 300 just proximate the distal end 302. For example, as illustrated in FIGS. 1-3, the steering wire 400 is coupled to the guide wire extension element 300 at a distal position 304.

Generally, the profile of the curvature of the guide wire extension element 300 is based on or is a function of the position at which the steering wire 400 is coupled to the guide wire extension element 300. For example, a steering wire that is coupled to the guide wire extension element closer in proximity to a distal end of the guide wire extension element (i.e., farther in proximity to tubular element 600) will facilitate a larger radius of curvature of the guide wire extension element than a steering wire that is coupled to the guide wire extension element farther in proximity to a distal end of the guide wire extension element (i.e., closer in proximity to tubular element 600).

In various examples, the guide wire extension element 300 and the steering wire 400 extend from the tubular element 600. As shown in FIG. 1, the guide wire extension element 300 and the steering wire 400 extend from a distal end 604 of the tubular element 600. In some examples, the tubular element 600 is hollow-bodied and operates in accordance with guide wire extension element 300 and the steering wire 400 to deliver an endovascular graft to a target site within a patient's vasculature. In some examples, the guide wire extension element 300 extends distally beyond the tubular element 600. In some examples, the guide wire extension element 300 extends a fixed distance distally beyond the tubular element 600. For example, the guide wire extension element 300 extends in the range of fifty (50) millimeters to one-hundred (100) millimeters, for example eighty-eight (88) millimeters, beyond the tubular element 600. However, it will be appreciated that the guide wire extension element 300 may extend some distance less than fifty (50) millimeters or more than one-hundred (100) millimeters beyond the tubular element 600 without departing from the spirit and scope of the disclosure.

In some other examples, the distance beyond which the guide wire extension element 300 extends from the tubular element varies or is variable or selective. That is, in some examples, the guide wire extension element 300 can be manipulated to extend a first distance beyond the tubular element 600 or a second distance beyond the tubular element 600. In some examples, the distance beyond which the guide wire extension element 300 extends from the tubular element is determined or selected based on the specific circumstances or need of the surgeon or operator. In some examples, as discussed in more detail below, membrane 500 is configured to stretch or deform to accommodate the selected configuration of the guide wire extension element 300 and steering wire 400.

As shown in FIG. 1, an olive 1000 is coupled to the distal end 302 of the guide wire extension element 300. In some examples, the olive 1000 is a soft and/or flexible tip positioned at the distal end 302 (or leading end) of the guide wire extension element 300. In some examples, the olive 1000 operates to help minimize vascular trauma and to enhance the positioning accuracy of the catheter assembly 200. In some examples, the steering wire 400 is coupled to the olive 1000. In one such example, the steering wire 400 is an integral component of the olive 1000. That is, in some examples, the steering wire 400 is coupled distal the guide wire extension element 300. In some examples, the olive 1000 has a lumen extending longitudinally therethrough. In one such example, the lumen extending through the olive 1000 is axially with the lumen extending through the guide wire extension element 300 such that the guide wire 700 is operable to extend therethrough and project distally thereof. That is, in some examples, the guide wire 700 extends though the lumen of guide wire extension element 300 and though the lumen of olive 1000.

As mentioned above, the endovascular graft delivery system 100 optionally includes a guide wire 700. The guide wire 700 may include but is not limited to tubes with lumens, solid rods, hollow or solid wires, hollow or solid stylets, metal tubes (e.g., hypotubes), polymer tubes, pull cords or tethers, fibers, filaments, electrical conductors, radiopaque elements, radioactive elements and radiographic elements. The guide wire 700 can be of any material and can have any cross-sectional shape including but not limited to profiles that are circular, oval, triangular, square, polygon-shaped or randomly-shaped.

In some examples, the catheter assembly 200 is operable to be guided along the guide wire 700. Specifically, the guide wire extension element 300 of the catheter assembly 200 is configured such that it can accommodate the guide wire 700 through its longitudinally extending lumen. Put differently, the guide wire extension element 300 is operable to receive the guide wire 700 and be guided therealong such that the catheter assembly 200 is guided along the guide wire 700 to a target site within a patient's vasculature.

However, given the sometimes tortuous nature of patients' vasculatures, the catheter assembly 200 operates in accordance with the guide wire 700 to navigate such tortuous vasculatures. Specifically, in some examples, the steering wire 400 is coupled to the guide wire extension element 300 such that the guide wire extension element 300 can be selectively deflected or steered. In some examples, where the guide wire 700 projects from the guide wire extension element 300 (or the olive 1000), selectively deflecting the distal end of the guide wire extension element 300 (or the olive 1000) causes the guide wire 700 to be deflected. Such deflections operates to facilitate the navigation of the guide wire 700 through the vasculature.

In addition, in some examples, the guide wire extension element 300 is selectively deflected or steered to facilitate proper alignment and orientation of an endovascular graft or other related medical device. That is, the guide wire extension element 300 can be deflected or steered to relocate, and/or pitch, and/or roll the endovascular graft or other related medical device such that it is properly oriented, aligned, and located within the patient's vasculature.

Accordingly, in some examples, the steering wire 400 facilitates the transitioning of the guide wire extension element 300 between an unsteered state (FIG. 1) and a steered state (FIG. 2). In the unsteered state the guide wire extension element 300 and the steering wire 400 extend substantially longitudinally parallel with each other. In the steered state, the steering wire 400 is tensioned such that the guide wire extension element 300 adopts a curvature along a portion of its length. That is, in the steered state, the guide wire extension element 300 and the steering wire 400 are not longitudinally parallel with one another. In some examples, the curvature adopted by the guide wire extension element 300 causes a separation of at least the intermediate portion 404 of the steering wire 400 from the guide wire extension element 300. That is, in the steered state, the guide wire extension element 300 deflects away from at least the intermediate portion 404 of the steering wire 400.

For example, referring now to FIG. 2, the catheter assembly 200 is illustrated in the steered state with the membrane 500 removed (for clarity purposes only). As illustrated, in the steered state, the guide wire extension element 300 is deflected away from the intermediate portion 404 of the steering wire 400 such that a void 1100 is created between a portion of the steering wire 400 and the guide wire extension element 300. In some examples, void 1100 is defined as extending between the guide wire extension element 300 and the steering wire 400. In some examples, the void 1100 is additionally defined as extending between the tubular element 600 and couple between the guide wire extension element 300 and the steering wire 400. In some examples, the void is generally defined between where the guide wire extension element 300 is separated from the steering wire 400. In some examples, the void is generally defined between where the guide wire extension element 300 is separated from the steering wire 400 to the extent that the separated area can be cannulated by another device (such as another guide wire or another medical device).

In some examples, steering of the catheter assembly 200 is facilitated by applying tension to steering wire 400. In some examples, steering of the catheter assembly 200 is facilitated by additionally or alternatively applying some distally directed force to guide wire extension element 300. In some examples, the application of tension and/or force to the steering wire 400 and/or guide wire extension element 300, respectively, causes a change in the relative lengths of the steering wire 400 and the guide wire extension element 300 projecting distally from the tubular element 600, thereby causing a portion of the guide wire extension element 300, such as the distal end of the guide wire extension element 300, to be deflected.

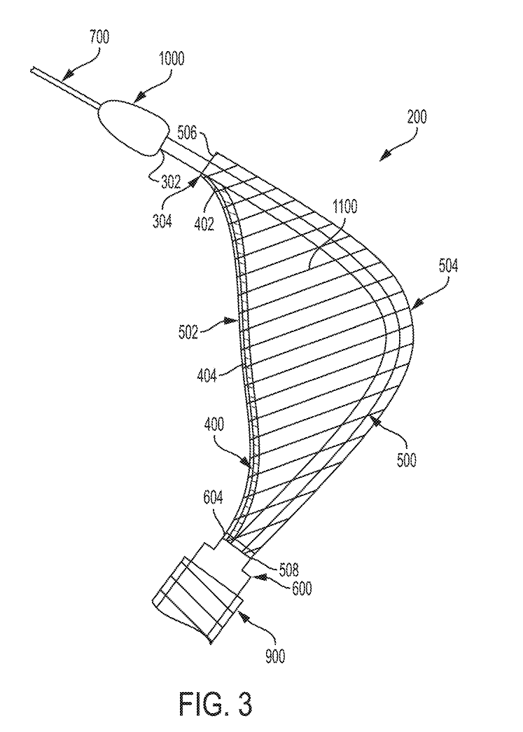

Referring now to FIG. 3, the catheter assembly 200 of FIG. 2 is illustrated with membrane 500 covering or otherwise spanning a portion of the void 1100. As illustrated in FIG. 3, the membrane 500 generally spans the void between the guide wire extension element 300 and the steering wire 400. In some examples, the membrane 500 completely covers the void 1100. In some examples, the membrane 500 covers a substantial portion of the void 1100. In some examples, the membrane 500 covers the void 1100 to the extent that the void 1100 cannot be penetrated or cannulated by another guide wire or instrument. That is, in some examples, the extent to which the membrane covers the void is measured in relation to the instruments the membrane operates to prevent from cannulating the void. Thus, in some examples, a membrane covers a substantial portion of a void where no instrument that is expected to encounter the membrane (such as another catheter or guidewire) could penetrate the void because any otherwise penetrable portion of the void not covered by the membrane is smaller than the size of the instrument that could otherwise penetrate that penetrable portion.

In some examples, a membrane covers a substantial portion of a void where the membrane covers at least 75% of the penetrable area of the void. It will be appreciated, however, that the membrane may completely cover the void. Additionally, it will be appreciated that the membrane may cover some portion of less than the entire void, as described above.

In some examples, membrane 500 is disposed about the guide wire extension element 300 and steering wire 400 such that the void 1100 cannot be cannulated or otherwise penetrated. That is, membrane 500 spans (or otherwise extends across) the void 1100 and operates to help prevent its penetration or cannulation by other objects, such as another guide wire or other instrument. By operating to help prevent such a penetration or cannulation, membrane 500 operates to help prevent against dislodgment of an endovascular graft or other issues.

In various examples, the membrane 500 extends along the guide wire extension element 300 and the steering wire 400. In some examples, the membrane 500 extends distally from the tubular element 600. In some such examples, a proximal end 508 of the membrane 500 is coupled to the tubular element 600. In some examples, the proximal end 508 of the membrane 500 is coupled to an exterior portion of the tubular element 600. In some examples, the proximal end 508 of the membrane 500 is coupled to an interior portion of the tubular element 600. In some examples, the proximal end 508 of the membrane 500 is coupled to the distal end 604 of the tubular element. In each of these examples, the membrane 500 extends distally along the guide wire extension element 300 and the steering wire 400 from where its proximal end 508 is anchored or coupled to the catheter assembly 200. As shown in FIGS. 1 and 3, the membrane 500 extends to a position situated between the distal end 302 and the distal position 304 of the guide wire extension element 300. However, in some examples, the membrane 500 extends to the distal end 302 of the guide wire extension element 300. In some other examples, the membrane 500 extends to a position distal to the distal end 302 of the guide wire extension element 300 (e.g., to an olive 1000, see below). In yet some other examples, the membrane 500 extends to a position proximal the distal position 304 of the guide wire extension element 300.

In some examples, the membrane 500 does not extend from the tubular element 600, but instead extends from a position distal to the tubular element 600. For example, as illustrated in FIG. 3, the proximal end 508 of the membrane 500 extends from a position 306 just distal to the distal end 604 of the tubular element 600. That is, in some examples, the membrane 500 is not coupled to the tubular element 600. In some examples, the membrane 500 is coupled to one or both of the guide wire extension element 300 and the steering wire 400.

In some examples, the membrane 500 is a resilient polymeric material such as PTFE, ePTFE, silicone, PET, nylon, pebax (or another suitable co-polymer), polyurethane, thermoplastic polyurethane or FEP imbibed ePTFE, or a suitable thermoplastic elastomer. Generally, the resilient polymeric material is thin, and/or elastic, and/or strong enough to be compatible with the device's operation without negatively impacting performance. In addition, in some examples, the resilient polymeric material is generally puncture resistant. In some examples, the resilient polymeric material can be formed in a manner (e.g., thickness) that facilitates a desired degree of puncture resistance.

It will be appreciated that by disposing a membrane 500 about the guide wire extension element 300 and the steering wire 400, the membrane 500 can operate to help prevent penetration or cannulation of the void 1100 created as a result of the guide wire extension element 300 and the steering wire 400 adopting a steered configuration (e.g., as illustrated in FIGS. 2 and 3). Specifically, by spanning a membrane 500 between the guide wire extension element 300 and the steering wire 400, the catheter assembly 200 of the present disclosure operates to cause any guide wire or other instrument that could otherwise cannulate the void 1100 to be deflected around catheter assembly 200. That is, any guide wire or other instrument that could otherwise cannulate the void 1100 is deflected around catheter assembly 200 as that guide wire or other instrument contacts membrane 500.

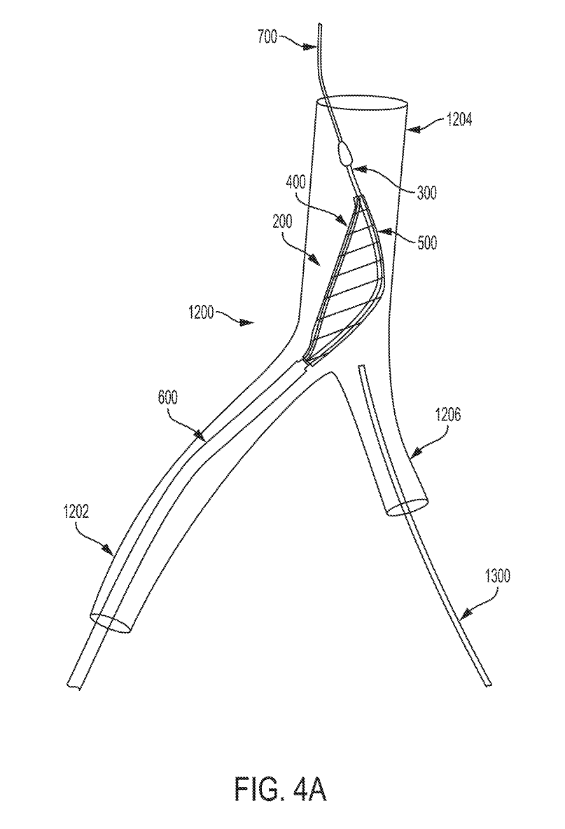

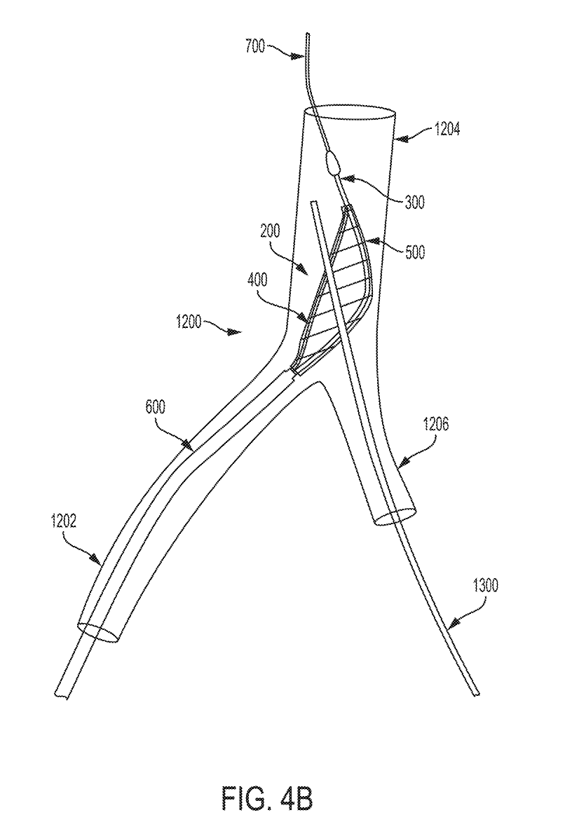

For example, referring now to FIGS. 4A and 4B, the anti-cannulation capabilities of the catheter assembly 200 are illustrated. In FIG. 4A, a catheter assembly 200 is shown extending into the interior of an expanded (or partially expanded) endovascular graft 1200. The catheter assembly 200 extends through a first leg 1202 into a trunk 1204 of the endovascular graft 1200. The catheter assembly 200 is illustrated in FIG. 4A in a steered position with membrane 500 covering the void formed between the guide wire extension element 300 and the steering wire 400. Also illustrated in FIG. 4A, a second guide wire 1300 is inserted through a second contralateral leg 1206 extending in a direction that would otherwise cause the guide wire 1300 to intersect with the membrane 500 of catheter assembly 200. However, as illustrated in FIG. 4B, the guide wire 1300 is deflected around catheter assembly 200 by membrane 500 such that guide wire 1300 does not cannulate or otherwise penetrate the void covered by membrane 500.

Specifically, as the guide wire 1300 is advanced distally into the endovascular graft 1200 along a path that intersects with the membrane 500 of the catheter assembly 200, the guide wire 1300 necessarily contacts the membrane 500. However, once the guide wire 1300 contacts the membrane 500, the membrane 500 causes the guide wire 1300 to deflect away from the catheter assembly 200, or at minimum prohibits the guide wire 1300 from penetrating or cannulating the void covered by the membrane 500.

By helping prevent the guide wire 1300 from cannulating the void covered by membrane 500, the catheter assembly 200 can be subsequently removed from the endovascular graft 1200 while the other guide wire 1300 remains inserted therein without the risk of an interference between catheter assembly 200 and guide wire 1300 that could lead to dislodgment of endovascular graft 1200 or other issues as a part of device delivery.

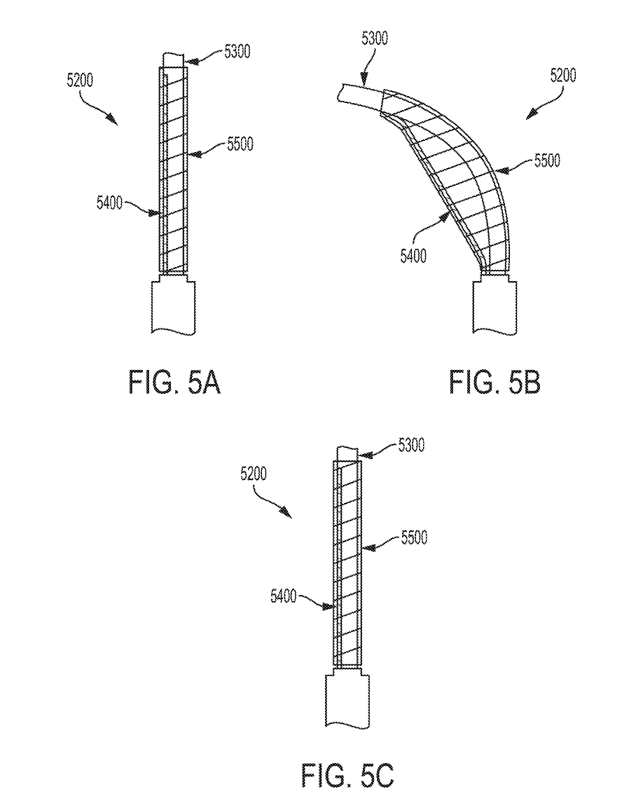

As discussed above, the membrane 500 operates to cover or span the void that is created when the guide wire extension element is deflected away from the steering wire. In some examples, the membrane is elastic in that it generally returns to its original shape and size after being stretched or otherwise deformed. For example, referring now to FIGS. 5A-5C, a catheter assembly 5200 is illustrated with an elastic membrane 5500. In an unsteered state (FIGS. 5A and 5C), the elastic membrane 5500 of the catheter assembly 5200 adopts a low profile configuration about the undeflected guide wire extension element 5300 and steering wire 5400 and generally contacts the guide wire extension element 5300 and the steering wire 5400 along their lengths. As illustrated in FIG. 5B, the elastic membrane 5500 is configured to elastically deform as the catheter assembly 5200 adopts the steered configuration. That is, the elastic membrane 5500 deforms or stretches to conform to the profile adopted by the steering wire 5400 and the deflected guide wire extension element 5300 when the catheter 5200 is transitioned to the steered state. Specifically, the elastic membrane 5500 deforms to accommodate the guide wire extension element 5300 as it separates and deflects away from the steering wire 5400. In some examples, as the guide wire extension element 5300 deflects, it contacts the elastic membrane 5500 and exerts a force upon the elastic membrane 5500 causing it to stretch and deform. In some examples, the elastic membrane 5500 adapts to dynamically accommodate the curvature of the guide wire extension element 5300 as it separates and deflects away from the steering wire 5400. That is, the elastic membrane 5500 deforms only to the extent necessary to accommodate the curvature adopted by the deflected guide wire extension element 5300.

In some examples, upon the catheter assembly returning to the unsteered state, the elastic membrane 5500 generally returns to the size and shape it adopted before the catheter assembly 5200 transitioned to the steered state. For example, upon transitioning back to the unsteered state, the elastic membrane 5500 returns to the size and shape it adopted before the guide wire extension element 5300 was deflected. Thus, while the elastic membrane 5500 is configured to deform to accommodate the shapes of the guide wire extension element 5300 and the steering wire 5400 when the catheter assembly is transitioned to a steered state, the elastic membrane 5500 is configured to generally return to its original size and shape when the catheter assembly is transitioned back to an unsteered state. Specifically, as the guide wire extension element 5300 returns to an undeflected configuration, it no longer exerts a force upon the elastic membrane 5500. Thus, the elastic membrane 5500 is not influenced to stretch or deform.

Thus, in some examples, the catheter assembly is configured such that when the catheter assembly is transitioned from an unsteered state to a steered state, the membrane is configured to transition between a first undeformed configuration and a second, different deformed configuration. In this example, when the catheter assembly is transitioned from the steered state back to the unsteered state, the membrane is configured to transition from the second, different deformed configuration back to (or substantially back to) the first undeformed configuration. In other words, in this example, the elastic membrane 5500 is configured to transition between a first undeformed configuration and a second, different deformed configuration without significantly plastically deforming.

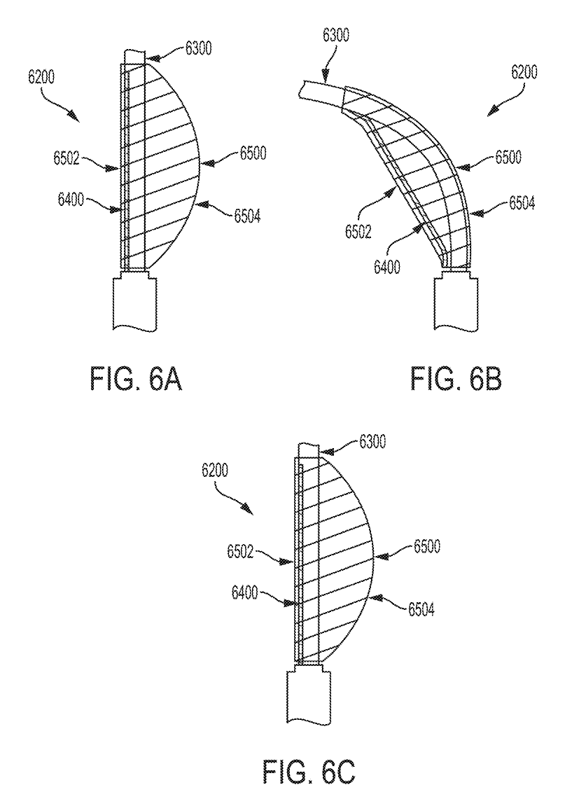

In some examples, a membrane having a designated size and shape is applied to the catheter assembly. In such examples, the membrane generally maintains its shape and size as the catheter assembly is transitioned between the unsteered state and the steered state. For example, referring now to FIGS. 6A-C, a catheter assembly 6200 is illustrated with a membrane 6500. As illustrated, membrane 6500 is pre-formed with at least one of its sides curved in accordance with the curvature the guide wire extension element 6300 is expected to adopt when the catheter assembly 6200 transitions to the steered state. That is, while the guide wire extension element 6300 and the steering wire 6400 are substantially parallel with one another and substantially non-curved, the membrane 6500 nevertheless is curved along at least one of its sides (e.g., side 6504). In some examples, the membrane 6500 is oriented such that its curved side 6504 is positioned proximate the guide wire extension element 6300. Likewise, the laterally opposing side 6502 of the membrane 6500 is generally positioned proximate the steering wire 6400. By properly orienting the membrane 6500 relative to the guide wire extension element 6300 and the steering wire 6400, the catheter assembly 6200 is smoothly transitioned between the unsteered and the steered states. That is, the membrane 6500 does not obstruct or help obstruct the deflection of the steering wire 6400 or the guide wire extension element 6300. Additionally, predisposing or pre-forming the membrane 6500 with a portion that is curved based on the curvature the deflected guide wire extension element 6300 is expected to adopt can help the guide wire extension element 6300 deflect freely and adopt the curvature without interference with or resistance by the membrane 6500.

Referring now to FIG. 6B, the catheter assembly 6200 is illustrated in the steered state. As illustrated, the curvature adopted by the guide wire extension element 6300 corresponds with the curved side 6504 of the membrane 6500. Thus, in some examples, the catheter assembly is configured such that when the catheter assembly is transitioned between the unsteered and steered states, the membrane is configured to maintain a first profile configuration.

When the catheter assembly 6200 is transitioned from the steered state to the unsteered state, the guide wire extension element 6300 returns to its original shape. That is, the guide wire extension element 6300 transitions back to a substantially non-curved profile. However, the membrane 6500 does not transition to a non-curved profile upon the catheter assembly 6200 transitioning back to the unsteered state. That is, after the catheter assembly 6200 returns to the unsteered state, the first side 6502 of the membrane 6500 maintains a generally non-curved profile and the second, laterally opposing side 6504 maintains its generally curved profile. For example, as illustrated in FIG. 6C, upon returning to the unsteered state, the catheter assembly 6200 generally returns to a configuration consistent with that of FIG. 6A. Specifically, upon returning to the unsteered state, the guide wire extension element 6300 is generally non-curved and extends substantially parallel with the steering wire 6400, while the membrane 6500 maintains at least one curved side 6504.

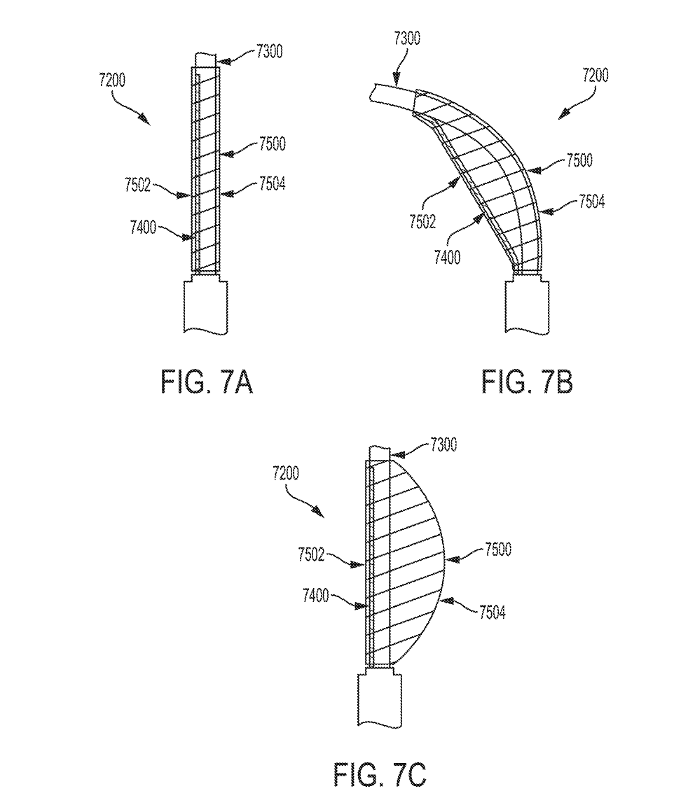

In some examples, the membrane is configured to plastically deform to accommodate the curvature adopted by the guide wire extension element as the catheter is transitioned to the steered state and the guide wire extension element deflects and exerts a force upon the membrane. Referring now to FIGS. 7A-7C, a catheter assembly 7200 is illustrated with a plastically deformable membrane 7500. In an initially unsteered state (FIG. 7A), the plastically deformable membrane 7500 of the catheter assembly 7200 adopts a low profile configuration about the undeflected guide wire extension element 7300 and steering wire 7400 and generally contacts the guide wire extension element 7300 and the steering wire 7400 along their lengths. As illustrated in FIG. 7B, the plastically deformable membrane 7500 is configured to deform as the catheter assembly 7200 adopts the steered configuration. That is, the plastically deformable membrane 7500 deforms or stretches to conform to the profile adopted by the steering wire 7400 and the deflected guide wire extension element 7300 when the catheter assembly 7200 is transitioned to the steered state.

Specifically, the plastically deformable membrane 7500 deforms to accommodate the guide wire extension element 7300 as it separates and deflects away from the steering wire 7400. In some examples, as discussed above, as the guide wire extension element 7300 deflects, it contacts the plastically deformable membrane 7500 and exerts a force upon the plastically deformable membrane 7500 causing it to stretch and deform. In some examples, the plastically deformable membrane 7500 adapts to dynamically accommodate the curvature of the guide wire extension element 7300 as it separates and deflects away from the steering wire 7400. That is, the plastically deformable membrane 7500 deforms only to the extent necessary to accommodate the curvature adopted by the deflected guide wire extension element 7300.

However, as illustrated in FIG. 7B the plastically deformable membrane 7500 deforms to conform to the profile formed by the steering wire 7400 and the deflected guide wire extension element 7300. That is, the plastically deformable membrane 7500 deforms such that first side 7502 maintains a low profile configuration proximate to the steering wire 7400 and such that the second, laterally opposing side 7504 deforms to accommodate and maintain a low profile configuration proximate to the deflected guide wire extension element 7300. Specifically, as the guide wire extension element 7300 deflects, it contacts the plastically deformable membrane 7500 and exerts a force upon the plastically deformable membrane 7500 causing it to stretch and plastically deform. In some examples, the plastically deformable membrane 7500 adapts to dynamically accommodate the curvature of the guide wire extension element 7300 as it separates and deflects away from the steering wire 7400. That is, the plastically deformable membrane 7500 deforms only to the extent necessary to accommodate the curvature adopted by the deflected guide wire extension element 7300.

While the plastically deformable membrane 7500 deforms to accommodate the curvature of the guide wire extension element 7300 as it separates and deflects away from the steering wire 7400, the plastically deformable membrane 7500 does not return to its original profile configuration after plastically deforming. Thus, upon the catheter assembly 7200 returning to the unsteered state, the plastically deformable membrane 7500 generally maintains its plastically deformed configuration (e.g., the shape and size adopted by the plastically deformable membrane 7500 in the steered state). Thus, in some examples, the catheter assembly is configured such that when the catheter assembly is transitioned from an unsteered state to a steered state, the membrane is configured to transition from a first undeformed configuration and a second, different deformed configuration. In this example, when the catheter assembly is transitioned from the steered state back to the unsteered state, the membrane is configured to maintain the second, different deformed configuration.

In various examples, as discussed above, the membrane is configured to span between the guide wire extension element and the steering wire such that a void formed therebetween (in both a steered or unsteered configuration) is protected against cannulation or penetration by another guide wire or instrument. In some examples, the membrane is formed by folding the membrane material around the guide wire extension element and the steering wire. In some examples, the membrane material is wrapped around both the guide wire extension element and the steering wire and reattached to itself.

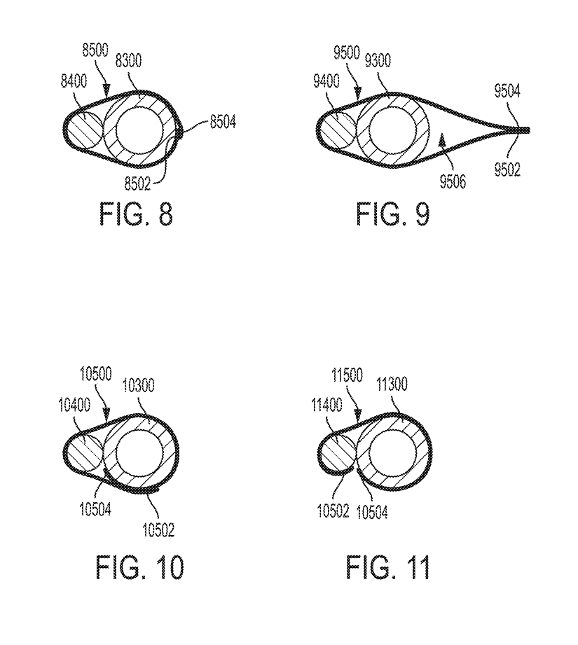

In one such example, the membrane material is wrapped around both the guide wire extension element and the steering wire and reattached to itself such that a first edge overlaps a second edge. For example, as illustrated in FIG. 8, a membrane 8500 is formed by winding a membrane material (as discussed herein) about a guide wire extension element 8300 and a steering wire 8400 and overlapping a first edge 8502 with a second edge 8504. In some examples, in forming the membrane 8500, the first edge 8502 is attached to one of the guide wire extension element 8300 and the steering wire 8400 and then wrapped around the guide wire extension element 8300 and the steering wire 8400 and reattached to itself. In some other examples, the membrane material is only attached to itself. That is, in such examples, the membrane material is not attached (or is not attachable) to the guide wire extension element 8300 or the steering wire 8400. It will be appreciated that FIG. 8 illustrates a cross-sectional view of a portion of the catheter assembly 8200 in an unsteered state. In some examples, the membrane material may be consecutively wrapped two or more times before the membrane material is reattached to itself. By consecutively wrapping the membrane material two or more times, the resulting membrane can be further reinforced.

In some examples, the membrane material is folded (or wrapped) around both the guide wire extension element and the steering wire and reattached to itself such that a first edge and a second edge are coupled together. For example, as illustrated in FIG. 9, a membrane 9500 is formed by folding a membrane material about a guide wire extension element 9300 and a steering wire 9400 and coupling together a first edge 9502 and a second edge 9504 such that the first edge 9502 and the second edge 9504 are aligned. In some examples, the first edge 9502 and the second edge 9504 are coupled together such that a gap 9506 is formed between a portion of the membrane 9500 and the catheter assembly 9200 (e.g., a gap 9506 is formed between a portion of the membrane 9500 and the guide wire extension element 9300). It will be appreciated that FIG. 9 is a cross-sectional view of a portion of the catheter assembly 9200 in an unsteered state.

In some examples, the membrane material is wrapped around both the guide wire extension element and the steering wire such that a first edge is attached to one of the guide wire extension element and the steering wire, while a second edge is attached to a portion of the membrane material. In some examples, the membrane material is wound around the guide wire extension element and the steering wire multiple times before the second edge is attached to the membrane material. In other examples, the membrane material is wound a single time before the second edge is attached to the membrane material. For example, as illustrated in FIG. 10, a membrane 10500 is formed by winding a membrane material about a guide wire extension element 10300 and a steering wire 10400. As illustrated, the membrane 10500 is formed by attaching a first edge 10502 to the membrane material and attaching a second edge 10504 to the guide wire extension element 10300. In this example, the second edge 10504 is attached to the guide wire extension element 10300, then the membrane material is wound around the guide wire extension element 10300 and the steering wire 10400, then the first edge 10502 is attached to the membrane material. It will be appreciated that FIG. 10 is a cross-sectional view of a portion of the catheter assembly 10200 in an unsteered state. As noted above, in some examples, the membrane material may be consecutively wrapped two or more times before the first edge is attached to the membrane material. By consecutively wrapping the membrane material two or more times, the resulting membrane can be further reinforced.

In some examples, the membrane material is wrapped around both the guide wire extension element and the steering wire such that a first edge is attached to one of the steering wire and the guide wire extension element while the second edge is attached to the other of the steering wire and the guide wire extension element. For example, as illustrated in FIG. 11, a membrane 11500 is formed by wrapping (or winding) a membrane material about a guide wire extension element 11300 and a steering wire 11400 and attaching a first edge 11502 with the steering wire 11400 and attaching a second edge 11504 to the guide wire extension element 11300. It will be appreciated that FIG. 11 is a cross-sectional view of a portion of the catheter assembly 11200 in an unsteered state.

In some examples, a pre-formed membrane is coupled with the catheter assembly. In some such examples, a membrane material is wrapped about a mandrel one or more times to create a membrane generally in the form of a tube having a lumen extending therethrough. Thus, in some examples, the membrane is longitudinally expansive and includes a lumen extending from its proximal end to its distal end. It will thus be appreciated that the membrane can be of any suitable material and can have any cross-sectional shape including but not limited to profiles that are circular, oval, triangular, square, polygon-shaped or randomly-shaped. In some examples, the membrane is attached to the catheter assembly such that the guide wire extension element and the steering wire pass through the lumen of the tube.

In some examples, the membrane is formed from a membrane material that is a long and narrow (such as a tape) that is consecutively wrapped around the mandrel such that each consecutive wrap progresses longitudinally along the axis of the mandrel (e.g., the membrane material is wrapped around the mandrel in a helical pattern). Accordingly, by progressively consecutive wrapping the narrow material around the mandrel, a longitudinally expansive, hollow membrane can be formed and subsequently attached to the catheter assembly. It will be appreciated that, when helically wrapping the membrane material around the mandrel, a first longitudinal edge of the membrane material generally consecutively overlaps a second longitudinal edge of the membrane material. Formation of a membrane in such manner provides versatility in not only the axial length of the membrane, but also provides versatility in the number of layers.

In some examples, the membrane material is alternatively or additionally wrapped about the mandrel one or more times without axially progressing along the mandrel. That is, the membrane material is wrapped such that a first longitudinal edge of the membrane material overlaps itself on each consecutive wrap. Likewise, the second longitudinal edge of the membrane material overlaps itself on each consecutive wrap. In one such example, the membrane material is wide (e.g., at least as wide as the desired longitudinal length of the membrane).

In some examples, the membrane is attached to the catheter assembly such that the guide wire extension element and the steering wire pass through the lumen of the tube. It will be appreciated that by consecutively wrapping the membrane material one or more times, a membrane with a designated number of layers can be created.

In some examples, a membrane can be formed though a continuous extrusion process. In some examples, a membrane can be formed through a blow molding process. In such embodiments, the membrane can be blow molded to adopt any desired shape and size.

In various examples, the membrane is coupled, fixed, attached, or otherwise fastened to the catheter assembly. In some examples, the membrane is coupled to the catheter assembly at a proximal and a distal end. For example, referring back now to FIG. 3, a catheter assembly 200 including a guide wire extension element 300, a steering wire 400 and a membrane 500 is illustrated. The catheter assembly 200 is illustrated in a steered configuration such that the membrane 500 spans between the deflected guide wire extension element 300 and the steering wire 400. In this illustrated example, the membrane can be coupled to the catheter assembly 200 at its distal end 506 and/or at its proximal end 508. In some embodiments, the membrane is coupled to the catheter assembly 200 at one or more locations where the distal end 506 contacts the catheter assembly and/or at one or more locations where the proximal end 508 contacts the catheter assembly. In some embodiments, the membrane 500 is coupled to the guide wire extension element 300 at its distal end 506 and/or its proximal end 508. In some examples, the membrane 500 is alternatively or additionally coupled to the steering wire 400 at its distal end 506 and/or its proximal end 508.

In various examples, the membrane is coupled, fixed, attached, or otherwise fastened to the catheter assembly along a longitudinal length of the catheter assembly. In some examples, the membrane 500 is coupled to the guide wire extension element 300 along a length of the guide wire extension element 300 (such as along a portion of the guide wire extension element 300 extending distally from the tubular element 600). For example, the membrane 500 may be coupled to the guide wire extension element 300 along a portion of the guide wire extension element 300 extending between the tubular element 600 and the distal end 302 of the guide wire extension element 300. In some examples, the membrane 500 is additionally or alternatively coupled to the steering wire 400 along a length of the steering wire 400 (such as along a portion of the steering wire 400 extending distally from the tubular element 600). For example, the membrane 500 may be coupled to the steering wire 400 along a portion of the steering wire 400 extending between the tubular element 600 and where the steering wire 400 couples to one of the guide wire extension element 300 and the olive 1000.

As mentioned above, in various examples, the membrane is coupled, fixed, attached, or otherwise fastened to the catheter assembly. In some examples, a shrink tube operates to couple the membrane to the catheter assembly, such as at a distal end and/or proximal end of the membrane. In some such examples, a shrink tube is placed over the distal end of the membrane and activated, causing a radial constrictive force to help hold the membrane in place and prevent its movement at that location relative to the catheter assembly. In some examples, a shrink tube is additionally or alternatively placed over the proximal end of the membrane and activated, causing a radial constrictive force to help hold the membrane in place and prevent its movement at that location relative to the catheter assembly. In some examples, the tube is activatable by way of heat. In some examples, the tube is activated chemically. It will be appreciated that any shrinkable tube that operates to help hold the membrane in place and prevent its movement at that location relative to the catheter assembly may be utilized without departing from the spirit or scope of the present disclosure.

In some examples, a shrinkable or activatable tape is utilized to couple the membrane to the catheter assembly. In some examples, the tape is wrapped around an end (such as a distal end and/or a proximal end) of the membrane. In some examples, the tape operates to help hold the membrane in place and prevent its movement at that location relative to the catheter assembly. In some examples, the tape operates to apply a sticking and/or radial constrictive force to help hold the membrane in place and prevent its movement at that location relative to the catheter assembly. In some examples, the tape can be activated. In some examples, the tube is activatable by way of heat. In some examples, the tube is activated chemically. It will be appreciated that any shrinkable or activatable tape that operates to help hold the membrane in place and prevent its movement at that location relative to the catheter assembly may be utilized without departing from the spirit or scope of the present disclosure.

In some examples, one or more fasteners (e.g., a nut, a bolt, a crimp, etc.) operate to couple the membrane to the catheter assembly. In some examples, an adhesive or bonding agent is utilized to couple the membrane to the catheter assembly. In some examples, the adhesive or bonding agent is incorporated into the membrane, guide wire extension element, and/or steering wire. In some examples, friction operates to couple the membrane to the catheter assembly. For example, the membrane may be manufactured such that it is stretched over the catheter assembly, and thereby exerts a radially constrictive force upon the catheter that operates to retain the membrane in a position relative to the catheter assembly.

It will be appreciated the various membrane coupling embodiments discussed herein may be combined in part or in whole without departing from the spirit or scope of the present disclosure.

In some examples, the tubular element 600 is configured to deliver an endovascular graft to a target area within a patient's vasculature. In some examples, the tubular element 600 has an endovascular graft disposed about a portion of its exterior. For example, referring again to FIG. 3, the tubular element 600 has an endovascular graft 900 situated about a portion of its exterior. In some examples, a selectively releasable sheath (not shown) operates to retain the endovascular graft 900 on the tubular element 600. In some examples, the selectively releasable sheath is a constraining sheath that compresses the endovascular graft about the exterior of the tubular element such that the catheter assembly retains a low profile during delivery of the catheter assembly to the target site within the patient's vasculature. In some examples, upon properly positioning the catheter assembly at the target site within the patient's vasculature, the sheath can be released such that the endovascular graft can expand and be anchored within the patient's vasculature (see FIG. 4).

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A steerable apparatus for insertion into a body comprising:
a guide wire extension element;
a steering wire; and
a membrane secured to the steering wire and the guide wire extension element.

2. The apparatus of claim 1, wherein the steering wire operates to cause a portion of the guide wire extension element to deflect away from the steering wire.

3. The apparatus of claim 2, wherein a void is formed between the guide wire extension element and the steering wire when the guide wire extension element is deflected away from the steering wire, the membrane covering the void to facilitate anticannulation of the void.

4. The apparatus of claim 3, wherein the membrane is elastic and is configured to stretch to accommodate a change in curvature of the guide wire extension element as it deflects away from the steering wire.

5. The apparatus of claim 3, wherein the membrane includes a pre-formed profile based on a profile that the guide wire extension element and the steering wire adopt when the steering wire operates to cause the guide wire extension element to deflect away from the steering wire.

6. The apparatus of claim 5, wherein the guide wire extension element is operable to deflect away from the steering wire and to conform to the pre-formed profile of the membrane.

7. The apparatus of claim 2, wherein a distal end of the steering wire is coupled to the guide wire extension element.

8. The apparatus of claim 2, wherein applying tension to the steering wire causes a portion of the guide wire extension element to deflect away from the steering wire.

9. The apparatus of claim 1, further including a tubular element, the steering wire and the guide wire extension element extending through a lumen of the tubular element and projecting distally from a distal end of the tubular element.

10. The apparatus of claim 9, wherein a length of the steering wire projecting distally from the distal end of the tubular element and a length of the guide wire extension element projecting distally from the distal end of the tubular element can be changed to cause the guide wire extension element to deflect away from the steering wire.

11. The apparatus of claim 1, wherein the membrane is coupled to one of the steering wire and the guide wire extension element.

12. The apparatus of claim 1, wherein the guide wire extension element has a lumen extending therethough, the lumen configured to accommodate a guide wire such that the guide wire extension element can be guided therealong.

13. The apparatus of claim 1, further including an olive coupled to a distal end of the guide wire extension element, a distal end of the steering wire being coupled to a portion of the olive.

14. The apparatus of claim 1, wherein the membrane is folded over the guide wire extension element and the steering wire and attached to itself.

15. The apparatus of claim 1, wherein the membrane is wound around the guide wire extension element and the steering wire and attached to itself.

16. The apparatus of claim 1, wherein the membrane is formed of a high strength film.

17. A method of manufacturing a steerable apparatus for insertion into a body comprising:
providing a steerable catheter delivery device including a guide wire extension element and a steering wire coupled to the guide wire extension element such that a force applied to the steering wire causes the guide wire extension element to deflect away from the steering wire to form a void between the guide wire extension element and the steering wire; and
coupling a membrane to the steerable catheter delivery device such that the membrane spans the void upon deflecting the guide wire extension element away from the steering wire, the membrane facilitating anticannulation of the void.

18. An endovascular delivery method comprising:
delivering a steerable guide wire assembly to a target site within a patient, the steerable guide wire assembly comprising a guide wire extension element, a steering wire, and a membrane in communication with the steering wire and the guide wire extension element;
radially displacing a portion of the guide wire extension element from the steering wire such that the guide wire extension element defines a curved portion forming a void between the curved portion and the steering wire, the membrane spanning the void to facilitate anticannulation of the void.

19. The method of claim 18, further comprising applying a tension to the steering wire to radially displace the guide wire extension element from the steering wire such that the guide wire extension element defines the curved portion.

20. The method of claim 19, further comprising releasing the tension to the steering wire to eliminate the separate of the guide wire extension element and the steering wire.
